(19) United States
(12) Patent Application Publication
(10) Pub. No.: US 2020/0045954 A1
(43) Pub. Date: Feb. 13, 2020

(54) NOVEL COMPOSITION FOR ORGAN PRESERVATION

(71) Applicants: Lindon Young, Philadelphia, PA (US); Robert Barsotti, Philadelphia, PA (US)

(72) Inventors: Lindon Young, Philadelphia, PA (US); Robert Barsotti, Philadelphia, PA (US)

(21) Appl. No.: 16/392,006

(22) Filed: Apr. 23, 2019

Related U.S. Application Data
(60) Provisional application No. 62/661,254, filed on Apr. 23, 2018.

(51) Int. Cl. A01N 1/02; C07K 5/087

(52) U.S. Cl. CPC A01N 1/021; C07K 5/0812

(57) ABSTRACT

The present invention relates to a peptide and solutions for preservation, perfusion, and/or reperfusion of an organ, especially the heart, for transplantation or after coronary angioplasty/coronary arterial bypass. The peptide contains the amino acid sequence of the sequence Phe-D-Arg-Phe-Amide (SEQ ID NO: 1), and the solution contains the peptide dissolved therein.

This application claims the priority of U.S. Provisional Patent Application No. 62/661,251, filed Apr. 23, 2018, which is incorporated herein by reference.

### FIELD OF THE INVENTION

The present invention relates to a peptide and solutions for preservation, perfusion, and/or reperfusion of an organ, especially the heart, for transplantation or after coronary angioplasty/coronary arterial bypass. The peptide contains the amino acid sequence of SEQ ID NO: 1; and the solution contains the peptide dissolved therein.

### BACKGROUND

Reperfusion of coronary blood flow to the ischemic heart following an acute myocardial infarction (MI), although necessary, may lead to myocardial ischemia/reperfusion (I/R) injury, resulting in cardiomyocyte death and compromised cardiac function (Hausenloy et al., J Clin Invest 2013, 123(1): 92-100). The major cause of I/R injury is due to reactive oxidative species (ROS), which damage the mitochondria that comprise up to one-third of the heart volume and is one of the key producers of ROS (Szeto, The AAPS Journal 2006, 8(2):E227-83) (FIG. 1). The generated ROS leads to the loss of mitochondrial membrane potential and opening of the mitochondrial permeability transition pore (MPTP), leading to cardiac contractile dysfunction and increased infarct size. As shown in FIG. 1, acute myocardial ischemia results in a decrease in pH due to the build-up of lactic acid from anaerobic conditions. The acidic conditions during ischemia prevent the opening of the mitochondrial permeability transition pore (MPTP) and cardiomyocyte hypercontracture at this time. Reperfusion results in washout of lactic acid, resulting in the rapid restoration of physiological pH, which releases the inhibitory effect on the MPTP opening, Ca²⁺ overload, and cardiomyocyte hypercontracture. The restoration of the mitochondrial membrane potential drives Ca²⁺ into the mitochondria, which can also induce MPTP opening and cardiac contractile dysfunction. Neutrophils accumulate in the infarcted myocardial tissue in response to the release of chemoattractants, and generate ROS.

There are currently no pharmacologic treatments that have been shown to clinically improve cardiac function and reduce infarct size in patients who have suffered from reperfusion-induced MI injury. Due to the role of mitochondria in l/R injury, treatment models targeting the mitochondria are a growing field in cardiovascular research.

Therefore, there remains a need for an agent improved quality that can protect the organ from ischemic injury, so that the organ can resume proper function after restoration of blood flow.

### SUMMARY OF THE INVENTION

An aspect of the present invention provides a tri-amino-acid peptide having the sequence Phe-D-Arg-Phe-Amide (SEQ ID NO: 1) (also referred to herein as “the tri-peptide”). The tri-peptide preferably has a molecular weight of 468 and protects organ tissues and cells from damage while the organ is isolated from the circulatory system or is experiencing decreased blood flow (ischemia).

Another aspect of the present invention provides a solution for protecting organs from ischemic damage. The solution containing the tri-peptide described above. Preferably, the tri-peptide is dissolved in a saline solution. The solution of the present invention may be used as a perfusion solution or a preservation solution. As a perfusion solution, it is pumped into the vasculature of the organ to protect the organ tissues and cells. As a preservation solution, it serves as a bathing solution into which the organ is submerged. Preferably, the organ is perfused with and submerged in the solution. Further, the present solution also serves as a reperfusion solution upon restoration of blood flow to the organ before or after ischemia.

A further aspect of the present invention include methods of using the solution of the present invention. These include methods for preserving an organ for transplantation, for protecting an ischemic organ from damage, for attenuating organ dysfunction after ischemia, and for protecting an organ from damage when isolated from the circulatory system.

Other aspects of the invention, including apparatuses, devices, kits, processes, and the like which constitute part of the invention, will become more apparent upon reading the following detailed description of the exemplary embodiments

### BRIEF DESCRIPTION OF THE DRAWINGS

The accompanying drawings are incorporated in and constitute a part of the specification. The drawings, together with the general description given above and the detailed description of the exemplary embodiments and methods given below, serve to explain the principles of the invention. In such drawings:

FIG. 1 is a chart showing the results of ischemia and of reperfusion.

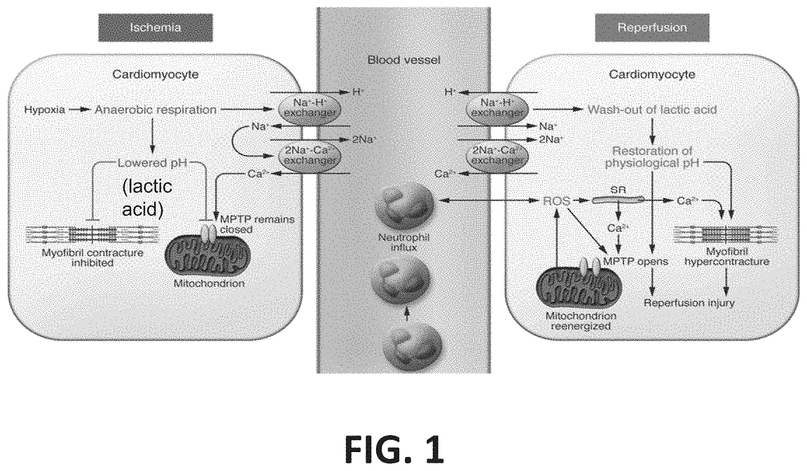

FIG. 2 is a flow diagram of the experimental protocol.

FIG. 3 are graphs shows representative tracings of the maximal rise of the left ventricular developed pressure (LVDP) [+dP/dtₘₐₓ] and the maximal decline of LVDP [−dP/dₜₘᵢₙ] for control I/R, Pretreat Tripep I/R, and Pretreat Tripep+Nlx I/R (left to right) hearts at 45 min reperfusion.

FIG. 4 is a graph showing time course of +dP/dtₘₐₓ for Groups I to V hearts.

FIG. 5 is a graph showing time course of −dP/dtₘᵢₙ for Groups I to V hearts.

FIG. 6 shows photographs of representative TTC stained heart sections from isolated perfused rat hearts.

### DETAILED DESCRIPTION OF THE PREFERRED EMBODIMENTS

The present invention provides a tri-amino-acid peptide having the sequence Phe-D-Arg-Phe-Amide (SEQ ID NO: 1) (also referred to herein as “the tri-peptide”). The tri-peptide preferably has a molecular weight of 468 and protects organ tissues and cells from damage while the organ is isolated from the circulatory system or is experiencing decreased blood flow (ischemia). The present inventor has discovered that the tri-peptide can exert protective effects in organs undergoing ischemia/reperfusion. The tri-peptide has also been found to attenuate ventilator induced diaphragmatic dysfunction.

The tri-peptide may be synthesized in accordance with any standard peptide synthesis protocol in the art. In one embodiment, the present synthetic peptides may be synthesized by use of a solid-phase peptide synthesizer (e.g., ABI433A peptide synthesizer, Applied Biosystems Inc., Life Technologies Corp., Foster City, Calif., USA) in accordance with the manufacturer's protocols.

Various functional groups may also be added at various points of the tri-peptide that are susceptible to chemical modification. Functional groups may be added to the termini of the peptide. In some embodiments, the function groups improve the activity of the peptide with regard to one or more characteristics, such as improving the stability, efficacy, or selectivity of the synthetic peptide; improving the penetration of the synthetic peptide across cellular membranes and/or tissue barrier; improving tissue localization; reducing toxicity or clearance; and improving resistance to expulsion by cellular pump and the like. Non-limited examples of suitable functional groups are those that facilitate transport of a peptide attached thereto into a cell, for example, by reducing the hydrophilicity and increasing the lipophilicity of the peptide, these functional groups may optionally and preferably be cleaved in vivo, either by hydrolysis or enzymatically, inside the cell. Hydroxy protecting groups include esters, carbonates and carbamate protecting groups. Amine protecting groups include alkoxy and aryloxy carbonyl groups. Carboxylic acid protecting groups include aliphatic, benzylic and aryl esters. In some optional embodiments, the carboxylic acid group in the side chain of the aspartic acid (D) of the present synthetic peptide is protected, preferably, by a methyl, ethyl, benzyl, or substituted benzyl ester.

The present invention also provides a solution for the preservation, perfusion, and/or reperfusion of an organ, especially the heart. The solution contains the tri-peptide. Preferably, the tri-peptide is present in the solution in a concentration of about 10 μM to about 100 μM, more preferably about 40 μM to about 60 M, most preferably about 50 μM.

In a preferred embodiment, the peptide inhibitor(s) are dissolved in a saline solution, preferably normal saline (0.9% NaCl). The peptide inhibitor(s) can also be dissolved in known preservation solution, such as Krebs-Henseleit solution, UW solution, St. Thomas II solution, Collins solution, Stanford solution, and the like. The solution may also contain one or more of sodium (Na⁺), potassium (K⁺), calcium (Ca²⁺), magnesium (Mg²⁺), glutamate, arginine, adenosine, manitol, allopurinol, glutathione, raffinose, and lactobionic acid in concentrations of about 4-7 mM, about 0.2-0.3 mM, about 108-132 mM, about 13-16 mM, about 18-22 mM, about 2-4 mM, about 0.5-1 mM, about 27-33 mM, about 0.9-1.1 mM, about 2.7-3.3 mM, about 25-35 mM, and about 80-120 mM, respectively. Na can be in the form of NaOH; K⁺ can be in the form of KCl and/or KH₂PO₄, most preferably at ratio of about 2-3.5 mM KCl and about 2-3.5 mM KH.sub.2PO.sub.4; Ca²⁺ can be in the form of CaCl₂); and Mg²⁺ can be in the form of MgCl2. The solution is preferably maintained at physiological pH of about 7.0-7.5, more preferably about 7.2-7.4.

The tri-peptide and solution of the present invention can be used during all phases of an organ, especially the heart, transplant, including, but are not limited to, 1) isolating of the organ from the donor (cardioplegic solution); 2) preserving the organ (hypothermic storage/transport); and 3) re-implanting the organ in the recipient (reperfusion solution). The protective effect may be before, during, or immediately after a surgical procedure on the organ, such as angioplasty, cardiac bypass or any procedure resulting in transient tissue ischemia.

In use, the tri-peptide may be placed in contact with the organ to protect it from ischemic injury. The organ may be placed in contact with the tri-peptide by soaking in the solution. Alternatively, the organ may be perfused with the solution containing the compound of Formula I. The contact of the tri-peptide with the organ may be iin vivo, in vitro, or ex vivo.

During perfusion or reperfusion, especially for the heart, it is preferred that the organ be perfused with the solution at a rate of about 1 mL/min for about 5 min. The perfusion rate can be varied, but it should not exceed about 25 mL/min. Overall, the perfusion rate should not be so high as to impose undue pressure on the vasculature of the organ.

The solution of the present invention can be prepared by 1) dissolving and diluting the peptide inhibitor(s) and the different constituents in distilled water; 2) adjusting the pH to about 7.2-7.4, e.g. with NaOH; and 3) sterilizing the solution, e.g., by filtering with a 0.2 μm filter. The sterilized solution is then kept isolated from contaminants in the environment.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in the example.

### Example 1

Male Sprague-Dawley rats (275-325 g, Charles River, Springfield Mass.) were anesthetized intraperitoneally (i.p.) with sodium pentobarbital (60 mg/kg) and anticoagulated with heparin 1000 units. Hearts were isolated and studied using a modified Langendorff heart preparation as previously described in U.S. Patent Application Publication Nos. 2016/0302406 and 2012/0141973, which are incorporated herein by reference.

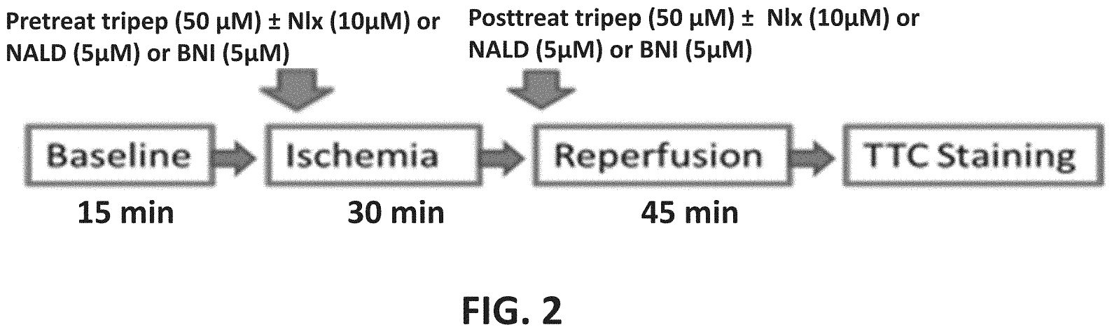

Tri-peptide (50 μM) was administered prior to ischemia (I) (pre-treatment, n=8) and at the beginning of reperfusion (R) in isolated perfused rat hearts subjected to global I(30 min)/R(45 min) in the presence/absence of naloxone (Nlx) (10 μM, n=8), or naltrindole (NALD) (5 μM, n=6) or nor-Binaltorphimine (BNI) (5 μM, n=6) and compared to untreated control I/R hearts (n=7) hearts. Control hearts did not receive tri-peptide, Nlx, NALD, or BNI. All treatments were prepared in Krebs' buffer and given during the last five minutes of baseline (pretreatment) and also prepared in plasma and given during the first five minutes of reperfusion via a syringe pump at 1 ml/min. All hearts were frozen at −20 C for 30 min, sectioned into 2 mm slices and incubated at 37 C in 1% 2,3,5-Triphenyltetrazolium chloride (TTC) to determine infarction size (see FIG. 2). The concentrations of opioid antagonists used in this study were based on previous reports (Wang et al., American Journal of Physiology 2001 280: H384-H391; Maslov et al., Medicinal Research Reviews 2016, 36(5):871-923).

The following groups of isolated perfused rat hearts were studied:
Group I: Control I/R hearts were subjected to 30 minutes global ischemia and then 45 minutes of reperfusion. All I/R groups were subjected to I(30 min)/R(45 min).
Group II: Pretreated Tri-pep+I/R hearts were first treated with the tri-peptide (50 μM). The treated hearts were then were subjected to ischemia and reperfusion with the tri-peptide (50 μM).
Group III: Pretreated Tri-pep+Nlx+IR hearts were first treated with the tri-peptide (50 μM) and Nlx (10 μM). The treated hearts were then subjected to ischemia and reperfusion with the tri-peptide (50 μM) and Nlx (10 μM).
Group IV: Pretreated Tri-pep+NALD+I/R hearts were first treated with the tri-peptide (50 μM) and NALD (5 μM). The treated hearts were then subjected to ischemia and reperfusion with the tri-peptide (50 μM) and NALD (5 μM).
Group V: Pretreated Tri-pep+BNI+IR hearts were first treated with the tri-peptide (50 μM) and BNI (5 μM). The treated hearts were then subjected to ischemia and reperfusion with the tri-peptide (50 μM) and BNI (5 μM).

All data in the figures are presented as means±S.E.M. ANOVA analysis using Student-Neuman-Keuls test was used to assess statistical difference in cardiac function and infarct size between Groups 1, II, III, IV, and V. Probability values of <0.05 were considered statistically significant.

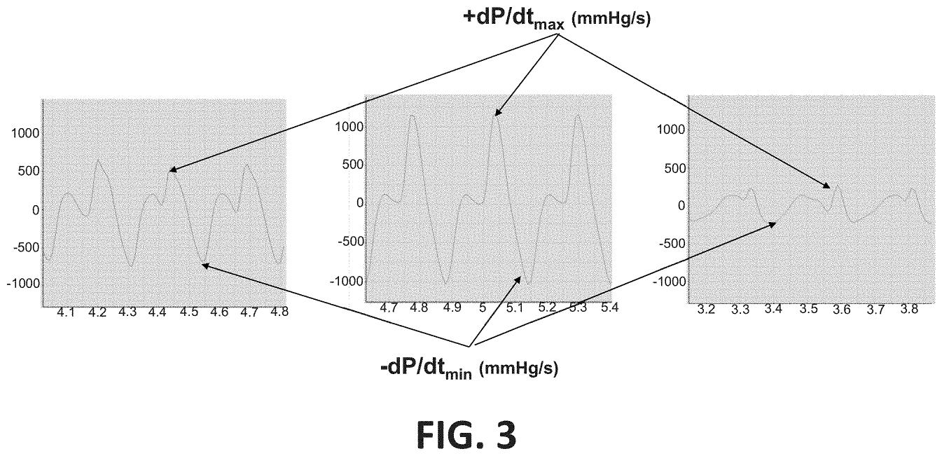

FIG. 3 shows the representative tracings of the maximal rise of the left ventricular developed pressure (LVDP) [+dP/dTmx] and the maximal decline of LVDP [−dP/d_{Tmin}] for control I/R, Pretreat Tripep I/R, and Pretreat Tripep+Nlx I/R (left to right) hearts at 45 min reperfusion.

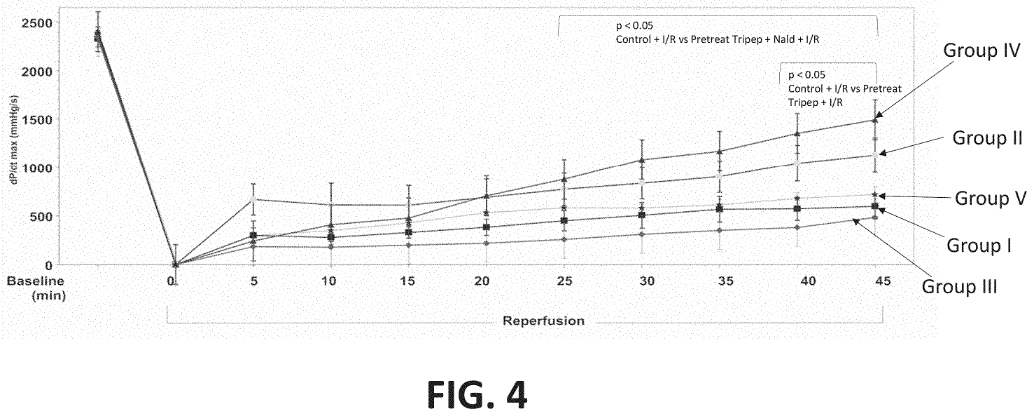

FIG. 4 shows the time course of +dP/dt max for hearts of Groups I-V.

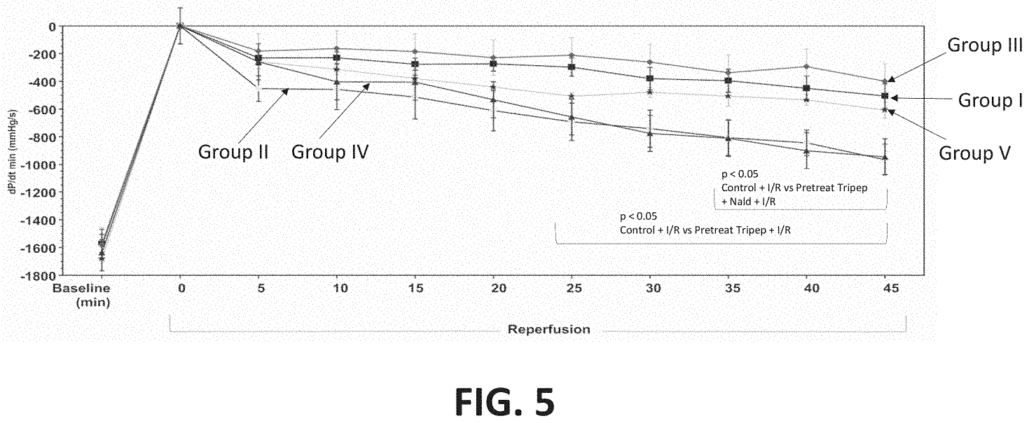

FIG. 5 shows time course of −dP/dt min for hearts of Groups I-V.

Table 1 shows cardiac function initial (baseline) and final (45 min R) values for hearts of Groups I-V ((*P<0.05; **p<0.01 vs. Control IR hearts). Left ventricular end systolic pressure=LVESP; Left ventricular end diastolic pressure=LVEDP; LVDP=LVESP−LVEDP.

TABLE 1

| Cardiac function | | | | | |
|---|---|---|---|---|---|
| & viability | Group I | Group II | Group III | Group IV | Group V |
| indices | (n = 7) | (n = 8) | (n = 8) | (n = 6) | (n = 6) |
| Initial LVESP | 103.3 ± 3.1 | 100.9 ± 3.1 | 96.7 ± 3.5 | 102.2 ± 4.4 | 98.1 ± 2.9 |
| (mmHg) | | | | | |
| Initial LVEDP | 7.9 ± 0.7 | 6.7 ± 0.8 | 5.2 ± 0.5 | 5.1 ± 0.94 | 5.05 ± 0.71 |
| (mmHg) | | | | | |
| Initial LVDP | 95.4 ± 2.8 | 94.2 ± 2.7 | 91.5 ± 3.2 | 97.1 ± 4.3 | 93.1 ± 2.7 |
| (mmHg) | | | | | |
| Final LVESP | 95.4 ± 3.5 | 105.6 ± 6.1 | 93.7 ± 5.5 | 94.6 ± 5.3 | 96 ± 6.01 |
| (mmHg) | | | | | |
| Final LVEDP | 64.1 ± 4.6 | 50.1 ± 7.4 | 70.2 ± 2.3 | 26.5 ± 6.2** | 60.5 ± 4.8 |
| (mmHg) | | | | | |
| Final LVDP | 31.4 ± 6.7 | 55.5 ± 6.0** | 23.5 ± 4.1 | 68.1 ± 7.5** | 35.47 ± 8.8 |
| (mmHg) | | | | | |
| Initial +dP/dtₘₐₓ | 2367.6 ± 68.1 | 2376.7 ± 75.1 | 2340.7 ± 56.8 | 2401.8 ± 101.0 | 2383.3 ± 44.5 |
| (mmHg/s) | | | | | |
| Final +dP/dtₘₐₓ | 596.6 ± 138.6 | 1126.8 ± 177.2** | 483.4 ± 79.6 | 1491.0 ± 194.3** | 718.1 ± 158.6 |
| (mmHg/s) | | | | | |
| Initial −dP/dtₘᵢₙ | −1612.5 ± 85.6 | −1590.5 ± 45.7 | −1581.1 ± 88.2 | −1637.3 ± 113.3 | −1681.9 ± 66.5 |
| (mmHg/s) | | | | | |
| Final −dP/dtₘᵢₙ | −505.6 ± 78.3 | 965.5 ± 112.1** | −399.2 ± 58.5 | −945.3 ± 133.9** | −608.7 ± 73.9 |
| (mmHg/s) | | | | | |
| Initial Coronary | 19.4 ± 2.2 | 17.7 ± 0.9 | 18.7 ± 1.9 | 17.9 ± 0.82 | 19.6 ± 1.0 |
| Flow (mL/min) | | | | | |
| Final Coronary | 7.8 ± 1.0 | 9.6 ± 1.5 | 6.7 ± 0.40 | 10.3 ± 0.75 | 7.8 ± 0.7 |
| How (mL/min) | | | | | |
| Initial Heart | 266.9 ± 9.5 | 287.8 ± 8.1 | 283.7 ± 5.8 | 263.5 ± 8.9 | 294.6 ± 8.4 |
| Rate (BPM) | | | | | |
| Final Heart Rate | 259.2 ± 11.2 | 276.3 ± 12.6 | 257.1 ± 8.5 | 258.0 ± 12.3 | 275.1 ± 39.9 |
| (BPM) | | | | | |
| Infarct Size | 38 ± 4% | 26 ± 1%* | 32 ± 3% | 20 ± 2%** | 30 ± 4% |

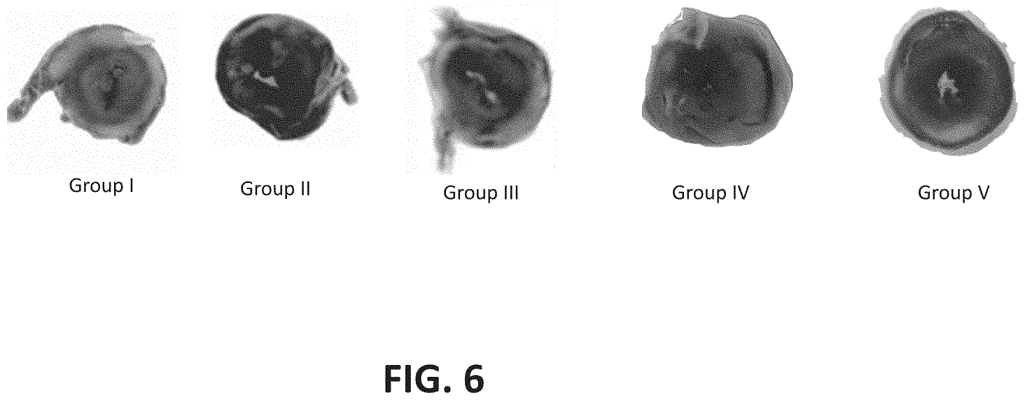

FIG. 6, shows the representative TTC stained sections from isolated perfused rat hearts of Groups I to V, respectively from left to right. Healthy heart tissue stained red, while infarcted heart tissue stained white.

Treatment with the tri-peptide significantly restored both post-reperfused cardiac function and reduced infarct size compared to untreated control I/R hearts. The improvement in post-reperfused heart function by tripeptide is most likely mediated through opioid kappa receptor activation since BNI not NALD blocked the cardio-protective effects of tri-peptide. The results from this study also suggest that the novel tri-peptide would mitigate clinical myocardial I/R injury to coronary angioplasty/bypass patients and organ transplant recipients.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A peptide comprising the amino acid sequence of SEQ ID NO: 1.

2. The peptide of claim 1, wherein a functional group is added to one of the amino acid.

3. A peptide consisting of the amino acid sequence of SEQ ID NO: 1.

4. The peptide of claim 1, wherein a functional group is added to one of the amino acid.

5. A solution for perfusion, preservation, and/or re-perfusion for organ preservation comprising the peptide of claim 1.

6. The solution of claim 5, wherein the peptide is dissolved in saline solution.

7. The solution of claim 5, further comprising potassium chloride.

8. The solution of claim 5, wherein the concentration of the peptide is about 10 μM to about 100 μM.

9. The solution of claim 5, wherein the organ is a heart.

10. The solution of claim 5, wherein the organ is a mammalian organ.

11. The solution of claim 5, wherein the organ is a human organ.

12. The solution of claim 5, wherein the mammalian organ is preserved for transplantation.

13. A method for preserving an organ for transplantation, protecting an ischemic organ, attenuating organ dysfunction after ischemia, or protecting an organ from damage after isolation from the circulatory system, said method comprising the step of contacting the organ with the solution of claim 5.

14. The method of claim 13, wherein the at least one peptide inhibitor is dissolved in saline solution.

15. The method of claim 13, further comprising potassium chloride.

16. The method of claim 13, wherein the concentration of the peptide is about 10 μM to about 100 μM.

17. The method of claim 13, wherein the contacting step comprises submerging the organ in the solution and/or perfusing the organ with the solution.

18. The method of claim 17, wherein the perfusing step takes place at a rate of less than about 20 mL/minute.

19. The method of claim 17, wherein the perfusing step takes place at a rate of about 1 mL/minute.

20. The method of claim 10, wherein the perfusing step lasts about 5 minutes.
